(19) United States
(12) Patent Application Publication
(10) Pub. No.: US 2009/0221945 A1
(43) Pub. Date: Sep. 3, 2009

(54) Liquid-Resistant Protection for an Extremity

(76) Inventors: Tommy Andersson, Norrköping (SE); Jan-Eric Sandell, Norrköping (SE)

Correspondence Address: HARNESS, DICKEY & PIERCE, P.L.C., P.O. BOX 8910, RESTON, VA, 20195, US

(21) Appl. No.: 11/793,518

(22) PCT Filed: Dec. 19, 2005
(86) PCT No.: PCT/SE2005/001981

§ 371 Date: Sep. 19, 2008

Related U.S. Application Data
(60) Provisional application No. 60/636,871, filed on Dec. 20, 2004.

(30) Foreign Application Priority Data
Dec. 20, 2004 (SE) 0403097-9

(51) Int. Cl. A61F 13/04

(52) U.S. Cl. 602/3

(57) ABSTRACT

A liquid-resistant protection for part of an extremity comprises a sleeve (1) of a waterproof polymer film, the sleeve (1) having an open end (2a). The liquid-resistant protection further comprises an adhesive layer (3) for sealing the sleeve (1) arranged on a section (3) of the outside of the sleeve (1) adjoining the open end (2a). A method of achieving a liquid-resistant protection around an extremity is demonstrated. A set of parts for forming a liquid-resistant protection for part of an extremity is furthermore demonstrated.

### TECHNICAL FIELD

The present invention relates to a liquid-resistant protection for an extremity according to the precharacterizing part of claim 1, a method of sealing such a liquid-resistant protection and a set of parts for producing a liquid-resistant protection.

### BACKGROUND OF THE INVENTION

Using a liquid-resistant protection for extremities, such as an arm or a leg, in order to protect open wounds, applied plaster of Paris or adhesive plasters, for example, on these parts of the body from wet and moisture when showering and/or bathing, for example, is already known.

EP-0 695 157 discloses such a liquid-resistant protection comprising a plastic sleeve with one open end and one end which has been sealed by heat sealing. In addition the liquid-resistant protection comprises a sealing strip which over its entire length comprises a tape, which is provided with an adhesive surface and a protective film covering this surface. The strip is furthermore of the same length as the sleeve and is fastened to the sleeve by way of a weld.

A further known liquid-resistant protection, which comprises a sock-shaped sleeve of a plastic film having an opening and an edge surrounding the opening, is described in U.S. Pat. No. 4,727,864. The protection further comprises a sealing strip of elastic material having an adhesive surface over its length. At one end the strip is fastened by ties on the outside and the edge of the sleeve. On the inside of the sleeve adjoining the edge there is also a smaller section of adhesive surface for fastening a part of the edge directly to the skin.

One problem of known liquid-resistant protections is that they are difficult to handle when fastening the liquid-resistant protection. A further problem is that they do not stay tight, especially when bathing, that is to say when the extremity is fully immersed in water.

### DISCLOSURE OF THE INVENTION

An object of the present invention is to provide an improved liquid-resistant protection, which entirely or partially eliminates the problems with the prior art.

The object is fully or partially achieved by a liquid-resistant protection, a method and a set of parts according to the respective independent claim.

Embodiments of the invention are set forth in the dependent claims and in the following description and the drawings.

According to a first aspect a liquid-resistant protection for an extremity is therefore provided comprising a sleeve of a waterproof polymer film, the sleeve having an open end. A distinguishing feature of the liquid-resistant protection is that an adhesive layer for sealing the open end of the sleeve is arranged on a section of the outside of the sleeve directly adjoining the open end.

Such an improved liquid-resistant protection provides an improved liquid-resistant protection which is easy to apply and which stays tight.

According to one embodiment the liquid-resistant protection may comprise two open ends. This embodiment is suitable for the liquid-resistant protection of an inner part of an extremity, such as an upper arm, for example.

According to one embodiment the liquid-resistant protection may comprise an open end and a closed end. This embodiment is suitable for the liquid-resistant protection of an outer part of an extremity, such as a hand or foot, for example.

According to one embodiment the liquid-resistant protection may further comprise an elongate sealing strip, one surface of which has an adhesive layer. Such a sealing strip affords the advantage that the sealing of the sleeve around an extremity can be improved. This allows an extremity with an injury, for example, to be fully immersed in water.

According to one embodiment the adhesive layer on the outside of the sleeve may consist of a double-sided adhesive tape. One advantage of such an adhesive layer is that it is easy to manufacture and apply to the sleeve.

According to one embodiment the section having the adhesive layer on the outside of the sleeve may run over basically half of the circumference of the sleeve. This affords the advantage that a simple and liquid-tight fastening of the sleeve is obtained when applying it to an extremity. In addition the adhesive layer is easy to apply to the sleeve during manufacture.

According to one embodiment the liquid-resistant protection may further comprise a venting valve for removing air included in the liquid-resistant protection. Such a venting valve means that an extremity, which has been provided with a liquid-resistant protection enclosing an injury, for example, can be fully immersed in water, without the water pressure causing the included air in the sleeve to destroy the liquid-tight sealing of the sleeve.

According to a second aspect a method is provided for forming a liquid-resistant protection around an extremity. The method involves introducing the extremity into a sleeve of waterproof polymer film, and tightening the open end of the sleeve around the extremity so that excess material forms a flap. The method further involves bending the flap towards the outside of the sleeve and causing the flap to adhere to the outside of the sleeve by means of an adhesive layer on a section of the outside of the sleeve that is situated directly adjoining the open end.

Such a method provides a simpler way of applying a liquid-resistant protection than with known liquid-resistant protections.

According to a third aspect a set of parts is provided for forming a liquid-resistant protection for an extremity. This set of parts comprises a sleeve of a waterproof polymer film, the sleeve having an open end, and an elongate sealing strip, one surface of which has an adhesive layer. A distinguishing feature of the set of parts is that an adhesive layer for sealing the open end of the sleeve is arranged on a section of the outside of the sleeve directly adjoining the open end.

Such a set of parts provides an improved liquid-resistant protection compared to known liquid-resistant protections, giving a liquid-resistant protection which remains tight, especially when bathing, when the extremity is fully immersed in water.

The set of parts may further comprise a venting valve for removing air included in the liquid-resistant protection. The advantage of such a venting valve has been discussed above.

A more detailed description of embodiments will now be given with reference to the drawings attached.

### BRIEF DESCRIPTION OF THE DRAWINGS

FIG. 1 is a schematic perspective view showing one embodiment of a liquid-resistant protection,

FIG. 2 is a schematic perspective view showing one embodiment of a liquid-resistant protection,

FIG. 3 is a schematic perspective view showing one embodiment of a liquid-resistant protection,

FIG. 4a-4b illustrates an example of how a liquid-resistant protection can be applied to a lower arm,

FIG. 5a-5b illustrates an example of how a liquid-resistant protection according to FIG. 2 or 3 can be applied to a lower arm,

FIG. 6 is a schematic perspective view showing one embodiment of a liquid-resistant protection comprising a venting valve.

### DESCRIPTION OF EMBODIMENTS

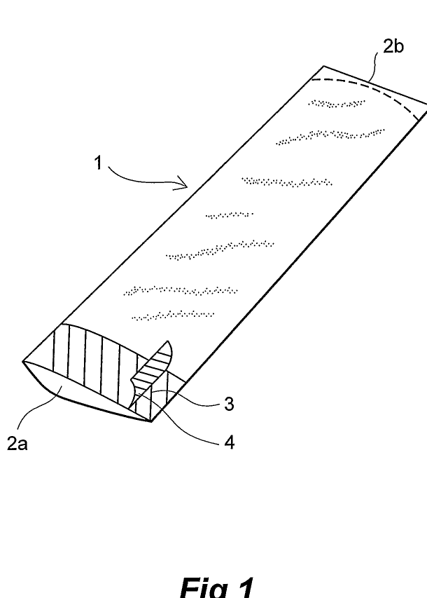

As is shown in FIGS. 1 to 3, an embodiment of a liquid-resistant protection may be formed by sealing one end of a plastic sleeve in order to form a bag 1 having an open end 2a and a closed end 2b. Unless otherwise specified, the sleeve 1 will consequently be referred to below as a bag 1.

FIG. 1 shows one embodiment of a liquid-resistant protection, which comprises an elongate, sleeve-like bag 1 of a waterproof polymer film. The polymer film may be composed of thin olefin plastic, such as ethylene plastics, propylene plastics or the like, or compounds thereof. The polymer film may further comprise additives such as ethylene butyl acrylate (EBA) in order to make the polymer film softer, more flexible and tougher. Other polymer materials and additives known to the person skilled in the art, which are suitable for forming a waterproof polymer film and which can be used in contact with the skin, may also be used. The bag 1 has an open end 2a through which an extremity, such as a part of an arm, a leg or a foot can be inserted. An adhesive layer 3 is arranged on a section 3 on the outside of the bag adjoining the open end. In the embodiment shown the section 3 with the adhesive layer 3 runs over basically half of the circumference of the bag. The adhesive layer 3 may also be covered with a protective film 4 maintaining the liquid-resistant protection. This protective film 4 may be designed so that it can easily be separated from the adhesive layer 3.

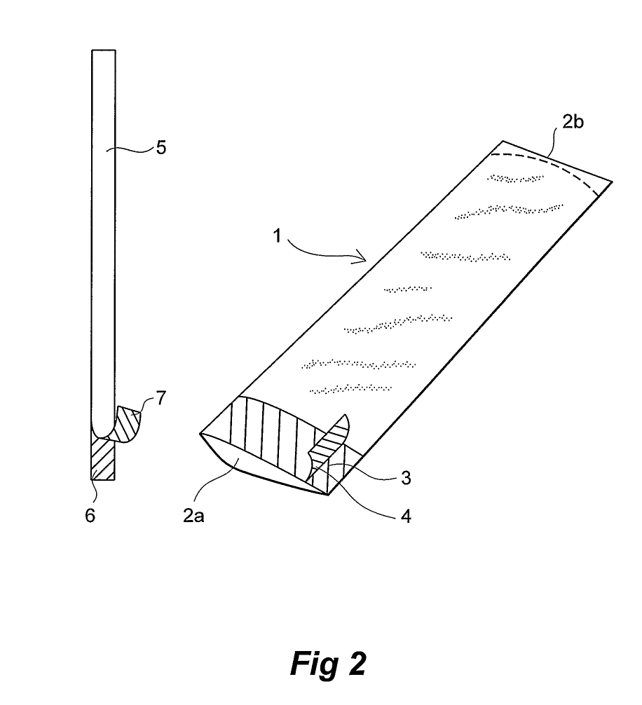

FIG. 2 shows a further embodiment of the liquid-resistant protection, which comprises a bag 1 similar to the embodiment of the bag 1 shown in FIG. 1. The embodiment according to FIG. 2 further comprises an elongate sealing strip 5, which like the bag may be composed of an olefin plastic. One surface 6 of the sealing strip 5 may have an adhesive layer 6. The adhesive layer 6 may also be covered with a protective film 7 maintaining the liquid-resistant protection. This protective film 7 can easily be separated from the adhesive layer 6. The sealing strip 5 may be integrated with the bag 1 or separate and packed in a set of parts together with the bag 1.

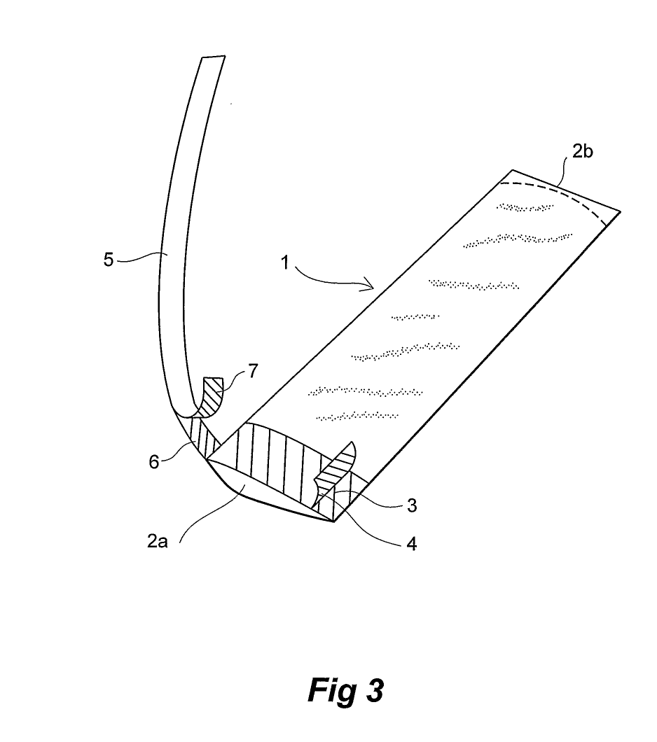

FIG. 3 shows one embodiment which is a variant of the embodiment shown in FIG. 2. The embodiment shown in FIG. 3 differs from that shown in FIG. 2 in that one end of the sealing strip 5 is fastened to the bag 1 adjoining the open end 2a. The sealing strip 5 may be fastened to the bag 1 by adhesive means, for example, or by means of a weld. Other ways of fastening such a sealing strip 5 known to the person skilled in the art may also be used.

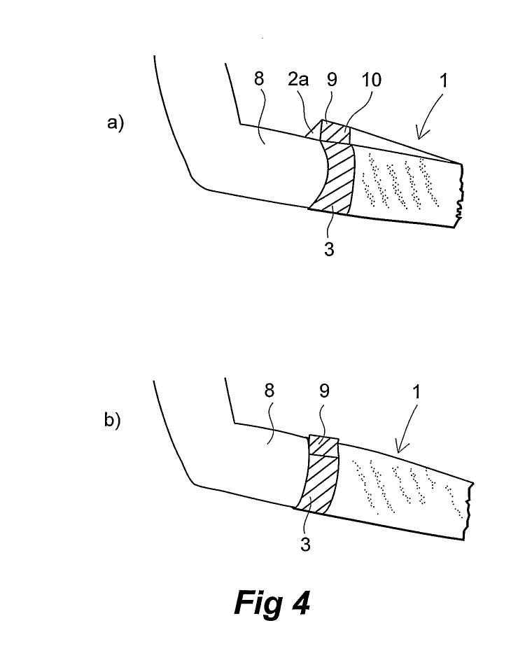

FIG. 4a-4b illustrate how the embodiment shown in FIG. 1 can be applied to a lower arm 8. As can be seen from FIG. 4a, a lower arm 8 has been inserted in through the open end 2a. The open end 2a of the bag 1 is then tightened around the lower arm 8, so that excess material forms a flap 9. A part of one side 10 of the flap comprises a part of or the entire section having the adhesive layer 3. The protective film 4 is then removed and the flap 9 is bent over so that the adhesive layer 3 is fastened to the part of the outside of the bag adjoining the open end 2a and resting flat against the skin (FIG. 4b). This method provides an easy way of fitting the liquid-resistant protection and at the same time a good protection against moisture, especially if contact is achieved between two sections of the adhesive layer, counteracting the formation of a duct through the fold.

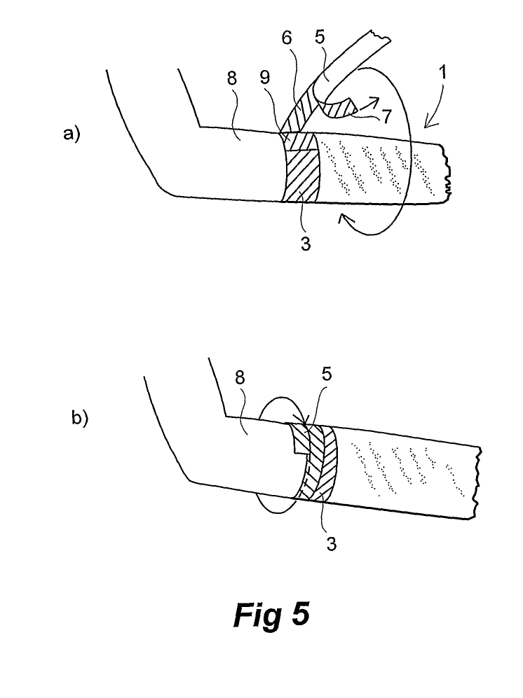

FIG. 5a-5b illustrates how a sealing strip 5 can be applied in order to further improve the protection against moisture. The bag 1 is first applied as described above in connection with FIG. 4a-4b. The protective film 7 is then separated from the sealing strip 5, which is in turn fastened to the outside of the bag 1 already applied in FIG. 4b, adjoining the open end 2a. The sealing strip 5 is then wrapped around the arm 8 so that it finally covers an area comprising a part of the section of the bag 1 at the open end 2a, which encloses the extremity and the skin bordering this section.

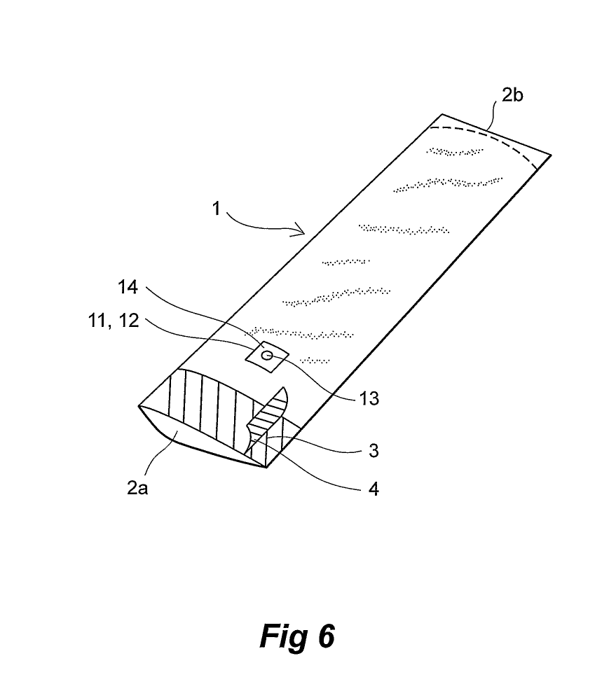

As shown in FIG. 6, one embodiment of the liquid-resistant protection may further comprise a venting valve 11. This venting valve 11 allows an extremity, which has been provided with a liquid-resistant protection enclosing an injury, for example, to be fully immersed in water. Without this venting valve 11 the water pressure would force included air inside the bag 1 out through the sealed open end 2a of the bag, which would result in destruction of the tight seal at the open end 2a of the bag 1. The venting valve 11 is suitably arranged on a section of the bag 1 adjoining the open end 2a of the bag 1, since the extremity provided with liquid-resistant protection is suitably immersed with that part of the extremity enclosed by the closed end 2b of the bag 1 being immersed first, followed by the part of the extremity enclosed by the open end 2a. The venting valve 11 may be fitted to the bag 1 before or after applying the liquid-resistant protection to an extremity as described above. One example of a venting valve 11 is a non-return valve 11, which allows included air in the bag 1 to flow out and which prevents liquid getting into the inside of the bag 1. The construction of such a non-return valve 11 will be familiar to the person skilled in the art and may comprise a valve body 12 having a through duct 13 in which, for example, a diaphragm, such as a rubber diaphragm, is designed to produce the function of the non-return valve. The valve body 12 may furthermore comprise a plate 14 through which the duct 13 runs in a direction at right angles to the plane of the plate 14. This plate 14 allows the valve to be fitted to the bag 1. For example, an adhesive layer may be arranged on one surface of the plate 14 for fitting to the bag 1. The venting valve 11 may furthermore be designed with means of penetrating the polymer film of the bag 1 and producing an opening over which the venting valve 11 can be fitted (not shown). In the absence of such means, the opening may be achieved by other means, such as a pen.

According to one embodiment the venting valve 11 may comprise an open duct 13 without a non-return valve function. In this embodiment the duct 13 may be sealed, for example, by means of a tape once the air has been forced out and before the section with the venting valve 11 is immersed in water.

According to one embodiment the adhesive layer 3 on the bag 1 may comprise a double-sided adhesive tape. It will be appreciated that other types of adhesive layer 4 may also be used, such as a layer of adhesive applied to the bag 1. It will be further appreciated that in various embodiments the section having the adhesive layer 3 may cover a greater or lesser part of the circumference and the length of the bag 1. The adhesive layer 3 must comprise of a part of the outer surface of the bag large enough to form a tight fold.

According to one embodiment the shape of the bag 1 is optional and may be adjusted to the contour of the extremity that is to be protected from liquid. An example of an optional shape of the bag 1 is a sock-shaped bag, which is intended for the liquid-resistant protection of a foot.

According to one embodiment the sealing strip 5 is of a length at least equal to the circumference of the extremity around which the sealing strip 5 is to be wound. This length permits a secure sealing of the bag 1 around the extremity, which provides a liquid-resistant protection which remains tight, especially when bathing, when the extremity is fully immersed in water.

A liquid-resistant protection is furthermore provided comprising a sleeve 1 of a waterproof polymer film, the sleeve 1 having two open ends 2a. An adhesive layer 3 for sealing the sleeve 1 is arranged in a section 3 on the outside of the sleeve 1 adjoining each of the open ends 2a. It will be appreciated that this embodiment may also comprise two sealing strips 5. Such a sleeve 1 having two open ends may be suitable for the liquid-resistant protection of the inner part of an extremity, such as an upper arm. The embodiment comprising a bag 1 having one open end 2a and one closed end 2b may be suitable for the liquid-resistant protection of an outer part of an extremity, such as a hand or foot. The sleeve 1 can thus be adapted to cover only the necessary part of an extremity.

1. Liquid-resistant protection for an extremity comprising a sleeve of a waterproof polymer film, the sleeve having an open end, characterized in that an adhesive layer for sealing the open end of the sleeve is arranged on a section of the outside of the sleeve directly adjoining the open end.

2. Liquid-resistant protection according to claim 1, comprising two open ends.

3. Liquid-resistant protection according to claim 1, comprising one open end and one closed end.

4. Liquid-resistant protection according to claim 1, further comprising an elongate sealing strip, one surface of which has an adhesive layer.

5. Liquid-resistant protection according to claim 1, wherein the adhesive layer is a double-sided adhesive tape.

6. Liquid-resistant protection according to claim 1, wherein the section having the adhesive layer runs over basically half the circumference of the sleeve.

7. Liquid-resistant protection according to claim 1, further comprising a venting valve for removing air included in the liquid-resistant protection.

8. Method of producing a liquid-resistant protection around an extremity, characterized in that the method involves introducing the extremity into a sleeve of waterproof polymer film, tightening the open end of the sleeve around the extremity so that excess material forms a flap, bending the flap towards the outside of the sleeve, and causing the flap to adhere to the outside of the sleeve by means of an adhesive layer on a section of the outside of the sleeve that is situated directly adjoining the open end.

9. Set of parts for forming a liquid-resistant protection for an extremity, the set of parts comprising a sleeve of a waterproof polymer film, the sleeve having an open end, and an elongate sealing strip, one surface of which has an adhesive layer, characterized in that an adhesive layer for sealing the open end of the sleeve is arranged on a section of the outside of the sleeve directly adjoining the open end.

10. Set of parts according to claim 9, further comprising a venting valve for the removal of air included in the liquid-resistant protection.
